(19) United States
(12) Patent Application Publication
(10) Pub. No.: US 2014/0187983 A1
(43) Pub. Date: Jul. 3, 2014

(54) Intravascular Guidewire with Hyper Flexible Distal End Portion

(71) Applicant: Volcano Corporation, San Diego, CA (US)

(72) Inventor: David Anderson, Temecula, CA (US)

(73) Assignee: Volcano Corporation, San Diego, CA (US)

(21) Appl. No.: 14/137,547

(22) Filed: Dec. 20, 2013

Related U.S. Application Data
(60) Provisional application No. 61/746,506, filed on Dec. 27, 2012.

(51) Int. Cl. A61B 5/00; A61M 25/09; A61B 17/12; A61B 5/0215; A61B 5/026

(52) U.S. Cl. CPC A61B 5/6851; A61B 5/0215; A61B 5/026; A61B 17/12113; A61M 25/09
USPC 600/486; 600/585; 600/505

(57) ABSTRACT

In one embodiment, a sensing guidewire for performing atraumatic intravascular physiologic measurements includes an elongated core wire and a sensor disposed at a distal end portion thereof. A flexure is disposed in the core wire proximal to the sensor housing. The flexure is substantially more flexible than regions of the core wire disposed on either side of the flexure, and enables a distal end portion of the guide wire to conform to and rest against a wall of vascular structure, such as an aneurism, without exerting an undue outward pressure thereon in response to making any contact with the wall.

### CROSS-REFERENCE TO RELATED APPLICATION

The present application claims the benefit of the filing date of provisional U.S. patent application Ser. No. 61/746,506 filed Dec. 27, 2012. The entire disclosure of this provisional application is incorporated herein by this reference.

### TECHNICAL FIELD

The present disclosure relates, in general, to intravascular devices, systems, and methods, and in particular, to intravascular guidewires with hyper flexible distal end portions, and methods for making and using them.

### BACKGROUND

When making physiologic measurements, such as blood pressure and/or blood flow measurements, in a relatively small vascular structure, such as an aneurism, a guidewire having a sensor located at or near its distal end that is capable of making such physiologic measurements can be inserted through a microcatheter and into the structure of interest. In some embodiments, the microcatheter and/or guidewire can be shaped to direct the distal end of the guidewire away from the aneurism wall. However, in some instances, the distal end portion of the microcatheter can be disposed near the aneurism wall. When this occurs, a guidewire extending from the microcatheter can come into traumatic contact with the aneurism wall. Depending on its axial rigidity, the guidewire may try to straighten itself, thereby applying an undesirable outward pressure on the wall of the aneurism, potentially resulting in trauma to or a puncture of the wall and resulting undesirable sequella.

Accordingly, a long felt but as yet unsatisfied need exists in the field of medical devices for intravascular guidewires, including guidewires having one or more sensors located at a distal end portion thereof, for performing physiologic measurements within aneurisms and similar thin-walled vascular structures that overcome the foregoing and other drawbacks of such devices.

### SUMMARY

In accordance with one or more embodiments of the present disclosure, intravascular guidewires with hyper flexible distal end portions are provided, together with methods for making them and using them in performing atraumatic blood pressure and flow assessments within aneurisms or other similar structures.

In one example embodiment, a sensing guidewire for performing atraumatic intravascular physiologic measurements comprises an elongated core wire and a sensor disposed at a distal end portion thereof. A flexure is disposed in the core wire proximal to the sensor housing. The flexure is substantially more flexible than regions of the core wire disposed on either side of the flexure, and enables a distal end portion of the guide wire to conform to and rest against a wall of vascular structure, such as an aneurism, without exerting an undue outward pressure thereon in response to making any contact with the wall.

In another embodiment, a method for using a guidewire device incorporating the novel guidewire above to effect measurement of physiological parameters, such as blood pressure and/or flow in, e.g., a physiological structure, comprises delivering a microcatheter into the structure such that a distal end of the microcatheter is disposed within the structure. The guidewire of the device is inserted into the microcatheter until a distal end of the guidewire is conterminous with the distal end of the microcatheter. The sensor is then exposed to a fluid within the structure, such as an aneurism, such that, in response to making any contact with a wall of the structure, a distal end portion of the guide wire conforms to, rests against and exerts a minimal contact force on the wall.

The scope of the present disclosure is defined by the claims appended hereafter, which are incorporated into this section by reference. A more complete understanding of the features and advantages of the novel guidewires of the disclosure and the methods for making and using them will be afforded to those skilled in the art by a consideration of the detailed description of some example embodiments thereof presented below, particularly if such consideration is made in conjunction with the appended drawings, wherein like reference numerals are used to identify like elements illustrated in one or more of the figures thereof.

### BRIEF DESCRIPTION OF THE SEVERAL VIEWS OF THE DRAWINGS

FIG. 1 is a partial cross-sectional elevation view of a conventional guidewire being introduced into an aneurism through a catheter.

FIG. 2 is a side elevation view of a conventional intravascular guidewire device to which the novel guidewires of the present disclosure have advantageous application.

FIG. 3 is an enlarged partial cross-sectional view of a distal end portion of an example embodiment of a guidewire having a hyper flexible distal end portion with a sensor thereon in accordance with the present disclosure.

FIG. 4A is a partial side elevation view of a conventional guidewire being supported at a selected distance proximal its distal end, showing a downward deflection, or “droop,” of a distal end portion thereof due to gravity.

FIG. 4B is a partial side elevation view of an example embodiment of a guidewire in accordance with the present invention being supported at the same selected distance proximal its distal end, showing the downward deflection, or droop, of the distal end portion thereof due to gravity.

FIG. 5 is a partial side elevation view of an example embodiment of a method and apparatus for evaluating the flexibility of the distal end portion of a guidewire in accordance with the present disclosure.

### DETAILED DESCRIPTION

In embodiments of the present disclosure, guidewires are provided for making physiologic measurements in a blood vessel or aneurism (where perforation or rupture is of significant concern) that reduce stress on the vessel/aneurism wall by placing one or more sensors located at the distal end portion of the guide wire with a hyper flexible core wire section proximal to the sensor housing, together with methods for making and using them.

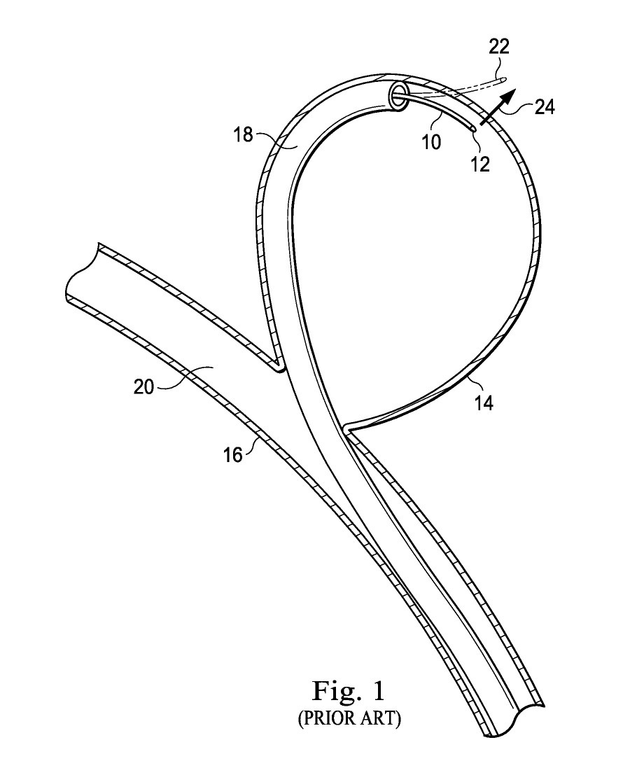

FIG. 1 is a partial cross-sectional elevation view of a conventional guidewire 10 having one or more sensors 12, such as a blood pressure sensor and/or a blood flow sensor, located at its distal end and being introduced into an aneurism 14 on a blood vessel 16, such as a vein or an artery, using a catheter 18 extending through the lumen 20 of the blood vessel 16 and into the aneurism 14.

As illustrated in FIG. 1, in some instances, the distal end of the microcatheter 18 can be disposed near the wall of the aneurism 14. When this occurs, a distal end portion of the guidewire 10 extending from the microcatheter 18 can come into contact with the wall of the aneurism 14. If the distal end portion of the guidewire 10 is relatively stiff, the distal end portion of the guidewire 10 may try to straighten itself into the configuration indicated by the dashed line 22, thereby causing the distal end portion of the guidewire 10 to apply an undesirable outward pressure on the wall of the aneurism 14, as indicated by the arrow 24, thereby potentially resulting in a perforation or rupture of the wall of the aneurism 14.

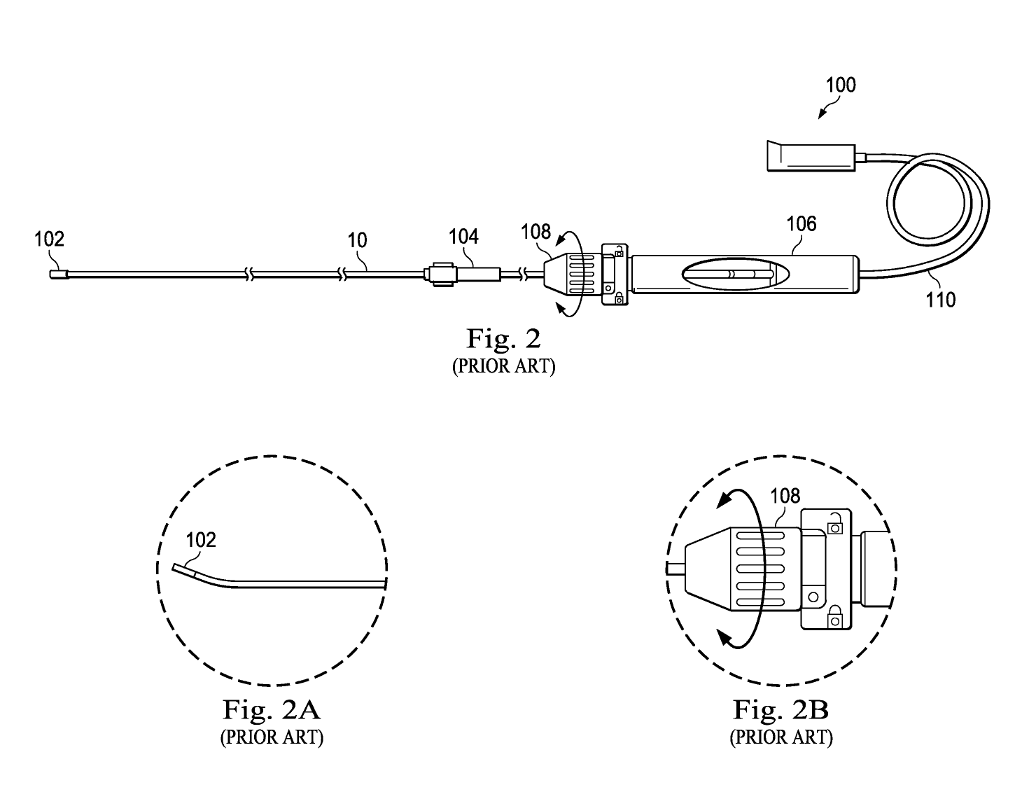

An example embodiment of a conventional guidewire device 100 capable of performing physiologic measurements and other intravascular procedures such as that described above is illustrated in FIG. 2, which illustrates a “ComboWire 0.0,” available from the Volcano Corporation, Rancho Cordova, Calif. In the particular embodiment illustrated in FIG. 2, the guidewire device 100 can include an elongated guidewire 10 having two sensors 12 located within a housing 102 disposed at its distal end. In one embodiment, the two sensors 12 can comprise, for example, a distal-end-mounted ultrasound transducer that transmits ultrasound waves and receives returned Doppler signals to measure blood flow, and a pressure sensor disposed immediately proximal of the flow sensor. In some embodiments, the pressure sensor can comprise a semiconductor diaphragm disposed over a sealed cavity and bordered by a flexible rim, for example, such as described in U.S. Pat. No. 6,106,476 to Corl et al. It should be understood that the device 100 is also available in models having only one sensor 12 at the distal end. In some embodiments, the housing in which the sensor(s) 12 can be from about 0 centimeters (cm), i.e., no housing, to about 3 cm in length. In one form, the housing is a substantially rigid hypotube section having a length between 1.5-2.5 mm, although shorter or longer housings may be utilized. In alternative embodiment, the housing is a flexible tubular member that may have a length greater than 3 mm up to 3 cm. Additionally, in some embodiments, the guidewire 10 can have no sensors 12 at its distal end, and other embodiments, the guidewire 10 can incorporate more than 2 sensors 12 at its distal end.

As illustrated in FIG. 2, in addition to the foregoing, the example guidewire device 100 can also include a distal end portion that is shapeable and/or radiopaque for visualization under fluoroscopy, a torqueing device 104, which can be used to rotate the guidewire 10 about its long axis, a connector body 106 configured to receive the proximal end of the guidewire 10 in a slide-in engagement, a connector body nose 108 for releasably locking the proximal end of the guidewire 10 in the connector body 106, and an electrical cable and connector plug 110 for connecting the signals from the two sensors to a monitor station (not illustrated) incorporating, e.g., a touch-screen display, a recordable CD drive, a printer, memory for storing sensor output data and other signal monitoring, displaying and recording components.

Examples of combination sensor guidewire devices 100 can be found in commonly owned U.S. Pat. Nos. 8,277,386 and 8,231,537, both to M. Ahmed et al., the disclosure of each of which is incorporated herein in its entirety.

A procedure such as discussed above in connection with FIG. 1 can be performed using a guidewire device 100 such as discussed above in connection with FIG. 2. Thus, in one example embodiment, the guidewire 10 of the device 100 is inserted into the aneurism 14 through a microcatheter, such as an Echelon-14 Microcatheter, available from Micro Therapeutics, Inc., Irvine Calif. The catheter 18 is initially delivered, which may be affected under fluoroscopy, into the aneurism 14 using a standard “front line” guidewire (not illustrated).

Once the microcatheter 18 is situated in the desired position, the front line guidewire is removed, and the guidewire 10 of the guidewire device 100 is inserted into the microcatheter 18 so that the distal end of the guidewire 10 is even with the distal end of the microcatheter 18. In one possible embodiment, the microcatheter 18 can then be moved proximally for a short distance, for example, about 10 mm, thereby exposing a corresponding 10 mm length of the distal end portion of the guidewire 10, without having to advance the guidewire 10 itself into the anatomy. Pressure, flow, and/or other measurements can then be made using, for example, the sensor(s) 12 disposed at the distal end portion of the guidewire 10. Thus, signals from the sensors 12 at the distal end portion are conveyed through the length of the guidewire 10 to the connector part 108 by thin conductive wires, and thence, through the cable and connector plug 100 to, for example, a monitoring station of the type described above.

In some procedures, the guidewire 10 and catheter 18 can be pulled back short distances and additional measurements taken. With proper initial positioning, measurements can be taken at many locations within the aneurism 14. At the completion of the procedure, the guidewire 10 can be withdrawn through the microcatheter 18 and out of the patient's body.

One drawback of conventional guidewires 10 is that they have relatively rigid distal end portions, i.e., the portion generally describing the distal-most 1-3 cm of the guidewire 10. As illustrated in FIG. 2, in the guidewire device 100 described above, both the pressure sensor and the Doppler transceiver are positioned in a tubular sensor housing 102 located at the distal end of the guidewire 10. In some embodiments, the sensor housing 102 can be approximately 2-3 mm long and can have a significant mass and rigidity, relative to the other components disposed at the distal end portion of the guidewire 10. Guidewires 10 conventionally comprise a flexible coil disposed concentrically about an elongated distal “core wire,” which forms a backbone of the guidewire 10. Thus, conventional guidewires 10 can have a distal core wire that includes a 1.5 centimeter (cm) long distal end “flat,” i.e., a flattened portion that is about 0.0017 inches (in.) thick.

A more flexible guidewire 10, such as a PrimeWire, available from Volcano Corp., typically has a distal end flat that is also about 1.5 cm long but only approximately 0.0009″ thick, which renders the distal end portion of the PrimeWire guidewire 10 relatively softer and more flexible than the embodiment above. The core wires of both guidewires have a similar distal core grind, i.e., a 0.0055 in. base core diameter that tapers down to a 0.0024 in. diameter over a 5 cm long taper, with a final cylindrical end portion grind that is about 0.0024″ in diameter and 2 cm long.

As those of some skill will understand, flexibility of the distal end portion of the guidewire 10 is based on several factors, including distal core wire grind diameter, distal flat length, distal flat thickness, distal flat width, and distal end portion coil design, material, and spacing. The respective mechanical stiffnesses of the distal core wire and distal flat are both functions of their Area Moments of Inertia (I) which, in the case of circular cross-sections, is calculated from the equation,

$$I = \frac{π {r}^{4}}{4} ,$$

where r is the radius of the circular cross-section, and in the case of rectangular cross-sections,

$$I = \frac{{WT}^{3}}{12} ,$$

where W is the width of the cross-section and T is its thickness.

From the above equations, it can be seen that, in the case of a circular cross-section, decreasing the diameter (a fourth power function), has a substantial impact on the flexibility of the distal end portion, and in the case of a rectangular cross-section, a decrease in the thickness T (a third power function) of the flat has a greater impact on the distal end portion flexibility than decreasing the width W. Generally, the width W of the flat (the less impactful dimension) is not controlled but is a result of the initial round profile cross-sectional area (derived from the final core grind diameter) and the thickness to which the core wire distal end portion is flattened. Nevertheless, some increase in the flexibility of the distal portion of the core wire can be obtained by locally reducing the width of the distal flat.

As those of some skill will understand, having a stable distal end portion is necessary when navigating, e.g., coronary anatomy. Some guidewires 10 are prepared for such use by putting a slight bend in the distal end portion of the wire, referred to as a “J-shape, as illustrated in the enlarged breakout view of FIG. 2. The “J” is typically 5-7 cm long and is bent at an angle of approximately 45 degrees. The actual size and angle of the bend can vary considerably, depending on the shaping method and the particular application at hand. The thicker distal end portion flat of a conventional guidewire 10 core wire, i.e., the mechanically flattened most distal portion of the core wire, typically about 1.5 cm long, makes the creation of the J-shape much more difficult. In the coronary and peripheral blood vessel anatomies, guidewires 10 can be “steered” by “torqueing,” i.e., rotating the direction which the J-shape points within the anatomy and advancing/retracting the guidewire 10, both typically under fluoroscopy, possibly using contrast injections to verify distal end portion location. With the sensors and sensor housing 102 disposed at the distal end of the distal end portion, their position relative to the body of the guidewire 10 and the J-shape needs to remain relatively stable, so that the guidewire 10 exhibits a predictable behavior when rotated or advanced or retracted axially. While it is possible to decrease the diameter of the core wire in the distal end portion or to decrease the thickness of the distal end portion flat, thus making the guidewire 10 more flexible and atraumatic, this would decrease the steerability of the guidewire 10 by making it difficult for the guidewire 10 to maintain a prescribed J-shape and destabilizing the handling of the guidewire distal end portion when steering inputs, i.e., torqueing, are applied.

However, in the aneurism assessment application described above, the guidewire 10 was delivered to the measurement location, viz., an aneurism 14, by passing it through a microcatheter 18 that had already been positioned using a frontline guidewire. Thus, as those of some skill will understand, it was not necessary to “steer” the guidewire 10 to its finally location. This is because the microcatheter 18 can have, for example, an internal lumen with a diameter of about 0.017 in., thus providing generous column support to the guidewire 10, which in some embodiments, can have an outer diameter of about 0.0145 in. As a result, the guidewire 10 can easily be advanced distally through the microcatheter 18 as long as the guidewire 10 has sufficient support, which can be provided by the core wire portions other than the distal end portion, along with adequate lubricity, which is also independent of distal end portion design, between the guidewire 10 and the catheter 18.

This delivery method therefore enables the creation of an improved guidewire 10 that has at least one flexible region, i.e., one or more “flexures,” disposed proximal to the distal sensor housing 102, thereby reducing the straightening force exerted on the wall of, e.g., an aneurism 14, when the guidewire 10 is exposed distally from the microcatheter 18 by the latter's withdrawal. This hyper flexible distal end portion design would, as discussed above, be detrimental to unaided steering of the guidewire 10 through, e.g., coronary anatomy, such as could occur, for example, in a typical percutaneous coronary intervention (PCI) procedure, but as discussed above, can be very beneficial in an atraumatic neuro/aneurism procedure, in which steerability of the distal end of the guidewire 10 is, as discussed above, of less importance.

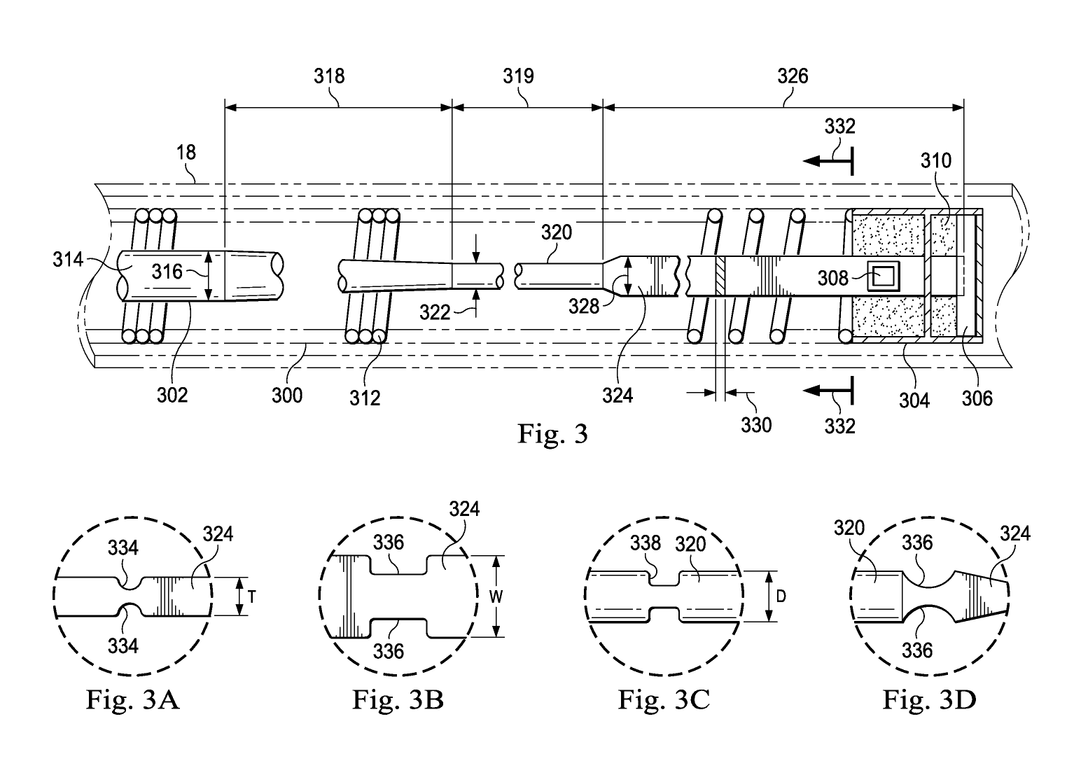

FIG. 3 is an enlarged partial cross-sectional view of a distal end portion of an example embodiment of a guidewire 300 having a hyper flexible distal end portion in accordance with the present disclosure, in which one or more flexures are incorporated into the core wire 302 posterior to a sensor housing 304 thereof to provide enhanced flexibility of the distal end portion.

As illustrated in FIG. 3, the example guidewire 300 comprises a core wire 302 and a sensor housing 304 disposed at the distal end of the guidewire 300. As discussed above, the sensor housing 304 can contain one or more sensors for sensing physiologic parameters within coronary, peripheral and neural locations in the body. In the particular embodiment illustrated in FIG. 3, for example, the sensor housing 304 is shown containing two sensors, viz., a blood flow sensor 306 mounted at the distal end of the guidewire 300, and a blood pressure sensor 308 mounted in a separate portion of the sensor housing 304 proximal to the flow sensor 306. In some embodiments, the sensors 306 and 308 can be at least partially embedded in matrix of a “potting” material 310. However, it should be understood that in other guidewire embodiments, other numbers, types and mountings of sensors could be implemented at or near, e.g., from approximately 0-3 cm, from the distal end of the guidewire 300, depending on the particular application at hand.

A coil 312 is disposed coaxially about the core wire 302. As discussed above, in some embodiments, the distal end portion of the coil 12 can be made radiopaque over a selected length to render it more visible under fluoroscopy. As illustrated in FIG. 3, in some embodiments, the spacing between the turns of the coil 312 at a distal end portion thereof can be increased to decrease the stiffness of a corresponding end portion of the guidewire 300. Alternatively, or in addition, the coil 312 can be wound from a source material having a diameter, e.g., from 0.001 in. to 0.003 in., so as to reduce the axial stiffness of the coil 312, and hence, the guidewire 300.

The guidewire 300 can have many possible configurations. However, for purposes of explication, a configuration corresponding to those discussed above is illustrated in FIG. 3. Thus, in the particular example embodiment of FIG. 3, the example core wire 302 includes a base or proximal core 314 having diameter 316 of 0.0055 in. The base core 314 tapers down over a distance 318 of 5 cm to a cylindrical portion 320 having a diameter 322 of 0.0024 in. and a length 319 of about 2 cm. As described above, a flat 324 is disposed at the distal end of the cylindrical portion 320. The example flat 324 has a length 326 of 1.5 cm, a width 328 (W) of 0.0027 in., and as discussed above, can have a thickness 330 (T) of from 0.0009 in. to 0.0017 in. It should be understood that the above configurations and dimensions are given by way of an example only, and that the core wire 302 can have many other configurations and dimensions, depending on the particular application at hand.

As discussed above, in order to render a distal end portion of the guidewire 300 hyper flexible, it is desirable to dispose one or more flexures within the core wire 302 proximal to the sensor housing 304, i.e., in a region of the core wire 302 disposed proximal to the arrows 332. The flexure in the core wire 302 should be substantially more flexible than regions of the core wire 302 disposed on either side of the flexure. As discussed above, this can be effected by reducing the area moment of inertia I in the region of the flexure relative to the area moment of inertia I of the adjacent regions of the core wire 302.

Thus, in one example embodiment, a substantial reduction in the flexibility of the distal end portion of the guidewire 300 can be effected by reducing the diameter, e.g., by grinding, of the cylindrical portion 320 of the core wire 302 to about 0.0015 in. If desired, the distal end portion of the cylindrical portion 320 could then be flattened to produce a flat of about 2 cm in length and about 0.0009-0.0017 in thickness.

This can be also be effected, for example, in the case of a flat 324 at the distal end of the core wire 302 by reducing at least one of the thickness T and/or the width W of the flat 324 in a region proximal to the sensor housing 304. As illustrated in the enlarged side elevation detail view A of FIG. 3, this can be effected, for example, by forming a pair of opposing notches 334, one each in the upper and lower surfaces of the flat 324, to reduce the thickness T of the flat 324 in that region, or, as illustrated in the top plan detail view B, by forming a pair of opposing notches 334, one each in the opposite sides of the flat 324, to reduce the width W of the flat 324 in that region, or by doing both.

As illustrated in detail side elevation view C, in some embodiments, it may be desirable to omit a distal flat 324, and to extend the cylindrical portion 320 of the core wire 302 to the distal end thereof. In this instance, a reduction in the area moment of inertia I in the desired region of the flexure relative to the area moment of inertia I of the adjacent regions of the core wire 302 can be effected, for example, by grinding one or more circumferential grooves 338 in the cylindrical portion 322 to reduce the diameter at the desired location of the flexure.

As illustrated in the top plan detail view D of FIG. 3, in yet another example embodiment, a pair of opposing notches 336 can be formed, e.g., by grinding, at the transition between the cylindrical portion 320 of the core wire 302 and a flat 324 at the distal end thereof.

As those of some skill in this art will understand, the above modifications to the core wire 302 can be effected in a number of known processes, including grinding, centerless grinding, conventional machining, micromachining, electrical discharge machining (EDM), pressing, and the like.

In some cases, it may be desirable to test guidewires with hyper flexible distal end portions made in accordance with the present disclosure at the prototype stage or during production to ensure that they exhibit the requisite degree of flexibility in their distal end portions.

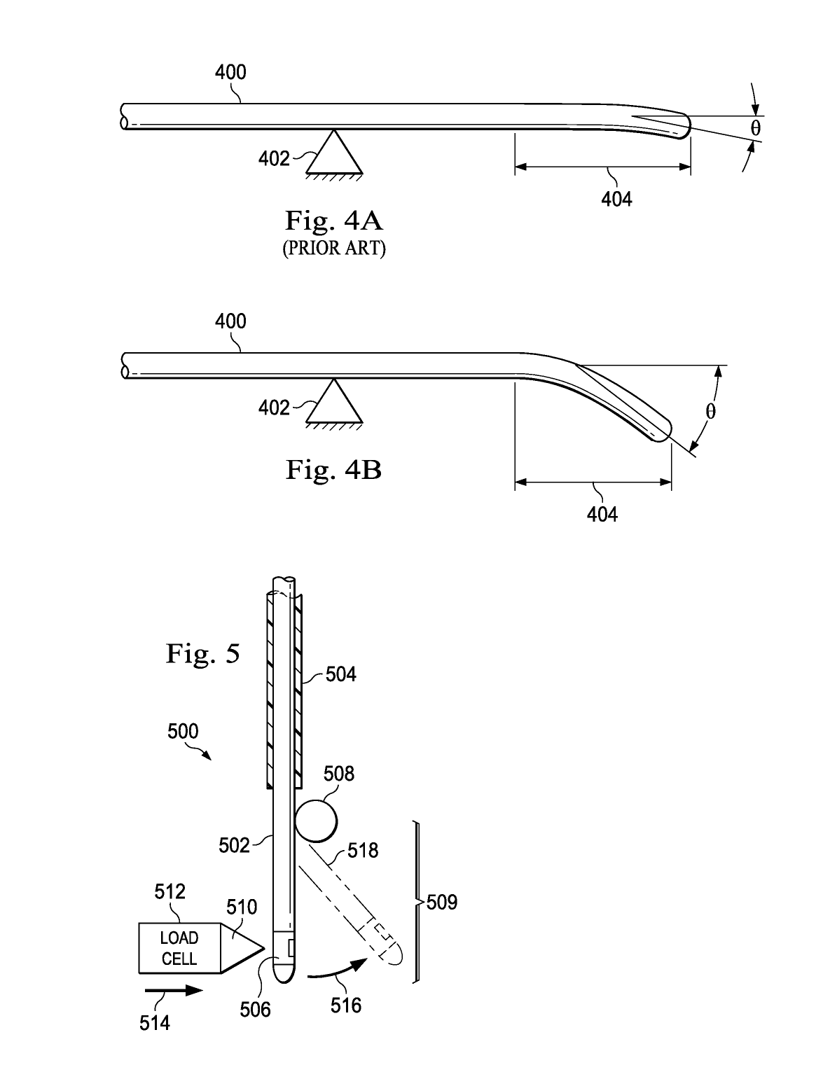

FIGS. 4A and 4B illustrate a simple type of test that can be performed to obtain a first-order evaluation of the flexibility of the distal end portion 404 of a guidewire 400. FIG. 4A is a partial side elevation view of a conventional guidewire 400 being supported by a fulcrum 402 or the like at a selected distance proximal to its distal end, showing a downward deflection, or “droop,” of the distal end portion 404 thereof due to gravity. As discussed above, due to the stiffness of the distal end portion intentionally incorporated therein in order to obtain the requisite degree of steerability of the distal end of the guidewire 400 within an anatomical vessel or chamber, the distal end portion 404 of the conventional guidewire 400 supported at a distance of 3 cm from the distal tip will droop or deflect under its own weight relative to the central axis of the guidewire by an angle Θ that is only from about 0 degrees to less than about 3 degrees.

FIG. 4B is a partial side elevation view of an example embodiment of a guidewire 400 in accordance with the present invention being supported at the same selected distance (3 cm) proximal its distal end, showing the downward deflection, or droop, of the distal end portion 404 thereof due to gravity. As illustrated, the guidewire is supported at a position proximal of the flexures or flex regions to allow gravity to act on the section of the guidewire distal of the flexure or flex regions. As illustrated in FIG. 4B and discussed above, by the provision of one or more flexures or flex regions in the core wire of the guidewire 400 proximal to its distal end, the hyper flexible distal end portion of the guidewire 400 can be configured to droop under its own weight relative to the central axis of the guidewire 400 through an angle Θ of from about 5 degrees to about 45 degrees, and preferably, within a range of 10 degrees to 35 degrees and still in a range of from about 15 degrees to about 25 degrees.

FIG. 5 is a partial side elevation view of an example method and apparatus 500 for determining the amount of force necessary to obtain a given deflection in the distal end portion of a guidewire 502. In FIG. 5, a test guidewire 502 is suspended vertically by a hypotube collar 504. This orientation is selected because, as discussed above, the hyper flexible distal end portions of the guidewires 502 are configured to have very weak “necks” proximal to their distal end portions 506, which, as discussed above, will tend to droop under their own weight if the guidewires were to be positioned horizontally. This vertical orientation makes it difficult to mount a fixture to a vertical tensile test machine, e.g., an Instron vertical test machine.

Accordingly, in the example method 500 illustrated, an arbor 508 is disposed adjacent to the guidewire 502 at a distance, e.g., about 10-30 mm, but in one example 20 mm posterior to the distal end portion of the guidewire 502 to create a fulcrum or pivot around which the guide wire 502 will bend when the distal end portion 506 is displaced laterally by moving, e.g., a wedge 510 disposed on a strain gauge or a load cell 512 in the direction of the arrow 514 and against the distal end portion 506. The load measured on the load cell 512 needed to bend the guidewire 502 through an angular displacement indicated by the arrow 516 and to the position indicated by the dashed lines 518 can be used as a measure of the flexibility/stiffness of the distal end portion of the guidewire 502.

As those of some skill appreciate, the designs for guidewires with hyper flexible distal end portions described herein can be applied to any measurement guidewire and used in any body locations, including many coronary, peripheral and neural locations in the body, where functional measurements are required.

The embodiments described above illustrate but do not limit the invention. It should also be understood that numerous modifications and variations are possible in accordance with the principles of the invention. Accordingly, the scope of the invention is defined only by the following claims and their functional equivalents.

What is claimed is:

1. A sensing guidewire, comprising:
an elongated core wire;
a sensor disposed at a distal portion of the guide wire; and
a flexure disposed in the core wire proximal to the sensor, the flexure being substantially more flexible than regions of the core wire disposed on either side of the flexure.

2. The guidewire of claim 1, wherein:
a distal end portion of the core wire has an area moment of inertia; and
the flexure comprises a region of the distal end portion having a reduced area moment of inertia.

3. The guidewire of claim 1, wherein:
a distal end portion of the core wire comprises a flat having a thickness and a width; and
the flexure comprises a region of the flat having at least one of a reduced thickness and/or a reduced width.

4. The guidewire of claim 1, wherein:
a distal end portion of the core wire comprises a cylinder having a diameter; and
the flexure comprises a region of the cylinder having a reduced diameter.

5. The guidewire of claim 1, wherein the sensor is disposed at a distal end of the guidewire.

6. The guidewire of claim 1, wherein the sensor comprises a pressure sensor or a flow sensor.

7. The guidewire of claim 1, further comprising a coil disposed coaxially about the core wire.

8. A guidewire device incorporating the guidewire of claim 1.

9. A method for using the guidewire device of claim 8, the method comprising:
delivering a microcatheter into the aneurism such that a distal end of the microcatheter is disposed within the aneurism;
inserting the guidewire of the device into the microcatheter until a distal end of the guidewire is conterminous with the distal end of the microcatheter; and
exposing the sensor to a fluid within the aneurism such that, in response to making any contact with a wall of the aneurism, a distal end portion of the guide wire conforms to, rests against and exerts a minimal contact force on the wall.

10. A method for making a guidewire, the method comprising:
providing an elongated core wire;
forming a flexure in the core wire proximal to a distal portion thereof, the flexure having a substantially greater flexibility than those of regions of the core wire disposed on either side of the flexure; and
coupling a sensor to the distal end of the guide wire.

11. The method of claim 10, wherein the forming comprises:
forming a distal end portion on the core wire, the distal end portion having an area moment of inertia; and
reducing the area moment of inertia of the distal end portion in a region proximal to the sensor.

12. The method of claim 10, wherein the forming comprises:
forming a distal end portion on the core wire, the distal end portion comprising a flat having a thickness and a width; and
reducing at least one of the thickness and/or the width of the flat in a region proximal to the sensor.

13. The method of claim 10, wherein the forming comprises:
forming a distal end portion on the core wire, the distal end portion comprising a cylinder having a diameter; and
reducing the diameter of the cylinder in a region proximal to the sensor.

14. The method of claim 10, further comprising disposing at least one sensor within a sensor housing.

15. The method of claim 14, wherein the at least one sensor comprises a pressure sensor or a flow sensor.

16. The method of claim 14, further comprising electrically connecting the at least one sensor to a proximal end of the guidewire with one or more conductive wires.

17. The method of claim 10, further comprising disposing a coil coaxially about the core wire.

18. A method for performing intravascular physiologic measurements in a vascular or coronary structure, the method comprising:
providing a catheter;
delivering a distal end of the catheter through a lumen of a blood vessel and into the structure;
providing a guidewire, including:
an elongated core wire;
at least one sensor disposed at a distal end portion of the core wire; and
a flexure disposed in the core wire proximal to the distal end portion, the flexure being substantially more flexible than adjacent regions of the core wire disposed on either side of the flexure;
inserting the guidewire into the catheter until a distal end of the guidewire is conterminous with the distal end of the catheter;
withdrawing the catheter from the structure such that a distal end portion of the guidewire is exposed within the structure; and
performing at least one physiologic measurement within the structure using the at least one sensor.

19. The method of claim 18, wherein the withdrawing comprises exposing a distal end portion of the guidewire within the structure such that, in response to making any contact with a wall of the structure, the distal end portion rests against the wall without undue outward pressure.

20. The method of claim 18, further comprising:
pulling the distal ends of the microcatheter and the guidewire back conjointly for a selected distance; and
performing at least one additional physiologic measurement within the structure using the at least one sensor.

21. A guidewire, comprising:
an elongated core wire; and
a flexure disposed in the core wire proximal to the sensor, the flexure being substantially more flexible than regions of the core wire disposed on either side of the flexure, wherein a distal end portion of the guidewire droops at an angle relative to a central axis of the guidewire of at least about 5 degrees when the guidewire is supported horizontally at a distance of 5 cm or less from a distal end thereof.

22. The guidewire of claim 21, wherein a distal end portion of the guidewire droops at an angle relative to a central axis of the guidewire of between about 5 degrees to about 45 degrees when the guidewire is supported horizontally.

23. A hyper flexible guidewire, comprising:
an elongated core wire having a proximal portion and a distal portion having a distal most tip, the proximal portion having a first area moment of inertia that is substantially greater than a second area moment of inertia of the distal portion, and
a tubular member covering at least a portion of the elongated core wire, wherein the distal tip droops 5 degrees or greater due to gravity when positioned horizontally and supported less than 5 cm from the distal tip.

24. The hyper flexible guidewire of claim 23, wherein the distal portion includes a rectangular cross section.

25. The hyper flexible guidewire of claim 23, wherein the distal portion includes a circular cross section.

26. The hyper flexible guidewire of claim 23, wherein the distal tip droops 5 degrees or greater due to gravity when positioned horizontally and supported 3 cm or less from the distal tip.

27. The hyper flexible guidewire of claim 23, wherein the distal tip droops 10 degrees or greater due to gravity when positioned horizontally and supported less than 5 cm from the distal tip.
